(19) United States
(12) Patent Application Publication
(10) Pub. No.: US 2020/0297962 A1
(43) Pub. Date: Sep. 24, 2020

(54) SUPER MASK RESPIRATOR SYSTEM HAVING A FACE MASK AND A SUB-PEAK INSPIRATORY FLOW BLOWER

(71) Applicant: AOK Tooling Ltd., Kowloon (HK)

(72) Inventors: Mei-Sheng Teng, Kowloon (HK); Steve Han, Huntington Beach, CA (US); Alex Stenzler, Long Beach, CA (US); William Ross, Las Vegas, NV (US)

(21) Appl. No.: 16/892,379

(22) Filed: Jun. 4, 2020

Related U.S. Application Data
(63) Continuation-in-part of application No. PCT/US19/30511, filed on May 3, 2019.
(60) Provisional application No. 62/697,777, filed on Jul. 13, 2018.

(51) Int. Cl. A61M 16/10; A61M 16/00; A61M 16/06; A61M 16/08; A61M 16/20

(52) U.S. Cl. CPC A61M 16/105; A61M 16/0066; A61M 16/0616; A61M 16/0833; A62B 23/025; A61M 16/0875; A61M 2016/0027; A61M 2205/0216; A61M 16/208

(57) ABSTRACT

A face mask includes an air-permeable body having a filter material having a proximal surface and a distal surface, the air-permeable body structurally configured for the proximal surface to cover the nose and mouth of a user when worn by the user, and a blower configured to generate a sub-peak inspiratory flow through the filter material.

### CROSS-REFERENCE TO RELATED APPLICATIONS

This application is a continuation-in-part of International Patent Application No. PCT/US19/30511 filed May 3, 2019, which claims priority to U.S. Provisional Patent Application No. 62/697,777 filed Jul. 13, 2018, the contents of which are each incorporated by reference herein in their entirety.

### BACKGROUND OF THE INVENTION

Face masks are often used as personal protective equipment in a variety of situations, such as during medical treatment or in dusty environments. Medical personnel, such as nurses and surgeons, often need to wear face masks when providing care to a patient. Such face masks are generally designed to filter airborne contaminants from the air being inhaled by the user in order to protect the user from inhaling pathogens and other contaminants, while also protecting people near the user from inhaling contaminants exhaled by the user. Industrial or consumer exposure to particles in the air can come from air pollution, pollens, fire, grinding, sanding, painting, etc. Such airborne contaminants may include aerosolized saliva, bacteria, viruses, dust from thousands of potential sources, or any other type of particle that can be suspended in air.

One conventional type of facemask with a high level of protection is the N95 mask, which refers to an efficiency rating determined by the National Institute for Occupational Safety and Health (NIOSH). The “N95” designation corresponds to a mask that blocks about 95% of particles that are 0.3 microns or larger. One common issue with N95 masks is that because of the high filtration rate, they generally increase the breathing effort required by the user to generate their normal unmasked inspiratory flowrate. Another issue with N95 masks and other types of filtration-based masks is that the moisture in the exhaled air is trapped inside the mask and this humidity also traps heat. This makes the masks uncomfortable to wear. Some N95 masks have exhalation valves to reduce some of the heat and moisture, but still require increased effort to breathe in. There are masks that depend on an external blower to flow fresh filtered gas into the mask to supply air for the user to breathe. These cool the air and lower the humidity, increasing comfort. Humans inhale in a sinusoidal pattern of flow so that there is a peak inspiratory flow that is greater than the mean flow during inhalation. To protect a user, a mask with a blower system either must have a reservoir to hold filtered air to meet the peak inspiratory flow or the flow of the blower itself must be greater than the peak inspiratory flow. If not, the user will inhale unfiltered air, placing them in danger of inhaling potentially harmful particles. Another general issue with filtration type masks is that the life of the mask is substantially determined by how long it takes for the filter material to become clogged with particles that are drawn to the mask during inhalation. When the material gets clogged to a level that makes breathing difficult, the mask needs to be replaced.

Thus, there is a need in the art for an improved respirator system that uses a blower to provide supplement inspiratory flow to a face mask to reduce breathing effort and improve comfort of the user without compromising the risk of inhaling unfiltered air.

### SUMMARY OF THE INVENTION

In one embodiment, a respirator system includes a face mask having an air-permeable body comprising a filter material having a proximal surface, a distal surface, and an opening, the air-permeable body structurally configured for the proximal surface to cover the nose and mouth of a user when worn by the user, and a blower unit having at least one filter, the blower unit being connected to the opening of the air-permeable body by a length of tubing and configured to generate a sub-peak inspiratory flow of filtered air through the tubing. In one embodiment, the blower is configured to generate only sub-peak inspiratory flow rates through a connector in the filter material. In one embodiment, a filter is attached proximal to the blower unit and distal to the tubing. In one embodiment, a filter is attached distal to the blower unit. In one embodiment, a first filter is attached distal to the blower unit and a second filter is attached proximal to the blower unit and distal to the tubing. In one embodiment, the tubing is detachable from the opening and replaceable with a one-way valve. In one embodiment, the blower is configured to generate a sub-peak inspiratory flow of between about 1 L min⁻¹ and 150 L min⁻¹. In one embodiment, the face mask further comprises a pressure sensor. In one embodiment, the blower has a speed configured as a function of air pressure measured by the pressure sensor during the user's inspiratory flow selected from the group consisting of: peak inspiratory flow, average inspiratory flow, and instant inspiratory flow. In one embodiment, the blower speed configuration is set to adjust the air pressure in the face mask that is a percentage of the absolute value of the negative air pressure measured during a user's peak inspiratory flow selected from the group consisting of: 10%, 20%, 30%, 40%, 50%, 60%, 70%, 80%, and 90%. In one embodiment, the blower speed configuration is set to adjust the air pressure in the face mask to a preset amount that is lower than the negative air pressure measured during a user's peak inspiratory flow while maintaining a net negative air pressure, the preset amount being selected from the group consisting of: minus 0.1 cmH₂O, 1 cmH₂O, 2 cmH₂O, 3 cmH₂O, 4 cmH₂O, and 5 cmH₂O. In one embodiment, the blower speed configuration is set to provide a lower air pressure limit of about minus 0.1 cmH₂O. In one embodiment, the face mask further comprises an inner seal attached to the proximal surface of the air-permeable body around the opening, the inner seal having a substantially circular shape configured to form an air-tight seal around a user's mouth and nose when worn. In one embodiment, the face mask further comprises an outer seal attached to the proximal surface of the air-permeable body, the outer seal forming a perimeter along the air-permeable body configured to form an air-tight seal around a user's face when worn.

In one embodiment, a respirator system includes a face mask having an air-permeable body comprising a filter material having a proximal surface, a distal surface, and an opening, the air-permeable body structurally configured for the proximal surface to cover the nose and mouth of a user when worn by the user; and a fan unit having at least one filter, the fan unit being connected to the face mask by a length of tubing and configured to generate an inspiratory flow of filtered air through the tubing between about 30 and 60 L min⁻¹. In one embodiment, the respirator system further comprises a Y-connector having a distal end and two proximal ends, wherein the distal end is connected to the length of tubing and the two proximal ends are each connected to an additional length of tubing that is connected to the face mask. In one embodiment, the fan unit comprises an outer side entry port and an inner air inlet, the outer side entry port having an opening larger than an opening of the inner air inlet. In one embodiment, the outer side entry port faces a direction orthogonal to a direction the inner air inlet faces. In one embodiment, the filter has a size that is substantially the same as the opening of the outer side entry port. In one embodiment, the filter comprises an elastomer gasket on an outer edge.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1 is a photo of a respirator system having a blower attached to a face mask according to one embodiment.

FIG. 2A is a front perspective view of a respirator system comprising a face mask connected to a blower having a proximal filter according to one embodiment. FIG. 2B is a cross-sectional diagram of the face mask shown in FIG. 2A, illustrating the passage of unfiltered air (solid lines) and filtered air (dashed lines). FIG. 2C is a rear perspective view of the face mask shown in FIG. 2A, illustrating the sealing strips for enhancing fit and air flow to the nose, mouth, and face of a user.

FIG. 3 is a cross-sectional diagram of the face mask shown in FIG. 2A illustrating the flow regimes of air during inspiration and exhalation. The top diagram depicts inspiratory flow that is less than blower flow, wherein the blower flow is capable of satisfying the inhaled air volume of a user and excess blower flow (if any) passes through the face mask filter material. The middle diagram depicts inspiratory flow that is greater than blower flow, wherein the inhaled air volume of a user that exceeds the amount supplied by the blower is supplemented by air drawn through the face mask filter material. The bottom diagram depicts expiratory flow, wherein the combined blower flow and a user's exhalation flow passes through the face mask filter material.

FIG. 4A is a front perspective view of a respirator system comprising a face mask connected to a blower having a distal filter according to one embodiment. FIG. 4B is a cross-sectional diagram of the face mask shown in FIG. 4A, illustrating the passage of unfiltered air (solid lines) and filtered air (dashed lines).

FIG. 5A is a front perspective view of a respirator system comprising a face mask connected to a blower having a proximal filter and a distal filter according to one embodiment. FIG. 5B is a cross-sectional diagram of the face mask shown in FIG. 5A, illustrating the passage of unfiltered air (solid lines) and filtered air (dashed lines).

FIG. 6 is a front perspective view of a face mask having an opening with an engagement for attaching a blower and a one-way valve according to one embodiment.

FIG. 7 is a front perspective view (left) and an exploded view (right) of a respirator system having a blower attached to a face mask according to one embodiment.

FIG. 8 is a front and rear perspective view of a fan unit (top) and a rear view of a power source (bottom) according to one embodiment.

FIG. 9 is a front perspective view of a fan and filter unit according to one embodiment.

FIG. 10 is a front perspective view of a fan unit according to one embodiment.

FIG. 11 is a cross-sectional side view of a fan and filter unit according to one embodiment.

### DETAILED DESCRIPTION OF THE INVENTION

It is to be understood that the figures and descriptions of the present invention have been simplified to illustrate elements that are relevant for a more clear comprehension of the present invention, while eliminating, for the purpose of clarity, many other elements found in respirators and face masks. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Ranges: throughout this disclosure, various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The present invention relates to respirator systems comprising face masks and blowers. The blowers provide a supplementary positive pressure inspiratory flow to the face masks to reduce breathing effort and to improve comfort by removing hot and humid exhaled air. Positive pressure also dislodges particles that are caught on the face masks, increasing their lifespan by removing clogs for a longer period of time. The blowers have an extended battery life that is able to meet the effort and comfort requirements of users with a lower flow demand on the blower. Users are thereby able to operate for long hours in a mobile environment without having to remove the respirator system in contaminated environments.

Referring now in detail to the drawings, in which like reference numerals indicate like parts or elements throughout the several views, in various embodiments, presented herein is a respirator system comprising a face mask connected to a sub-peak inspiratory flow blower.

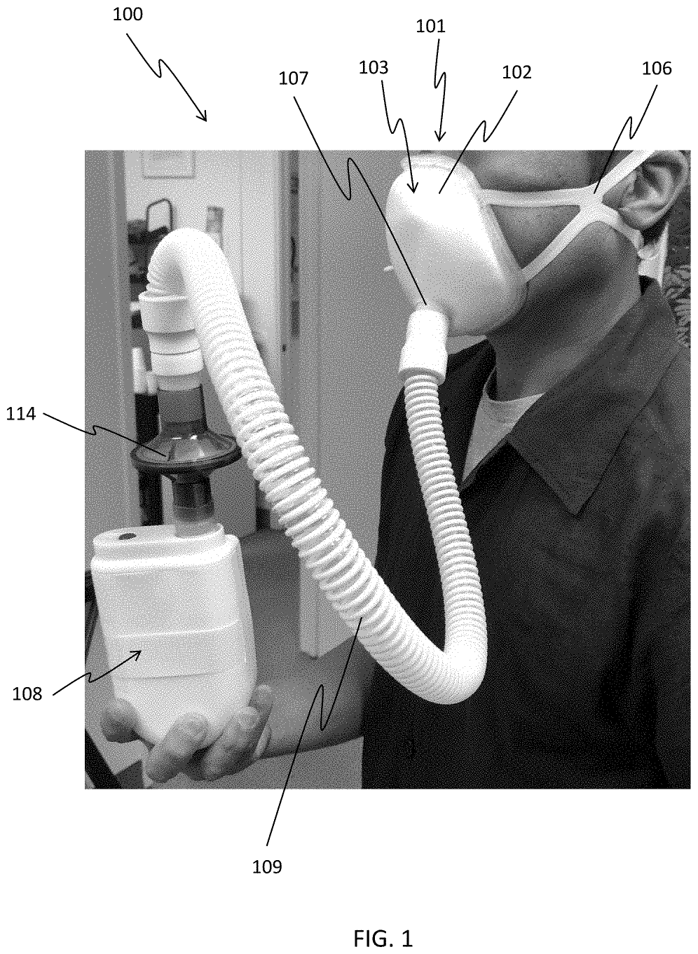

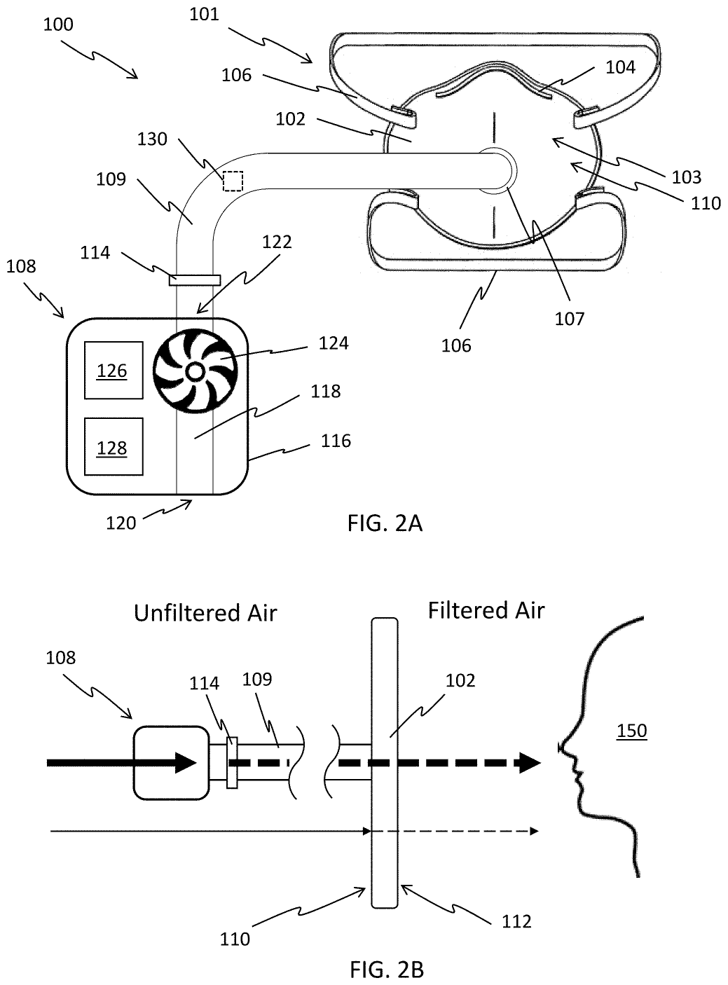

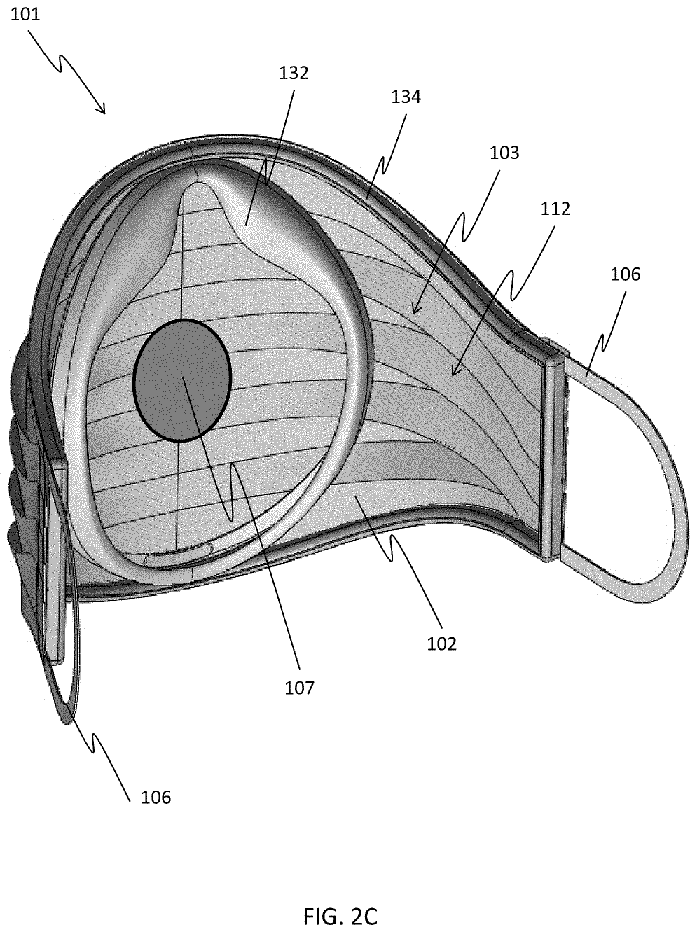

With reference now to FIG. 1, FIG. 2A, FIG. 2B, and FIG. 2C, a respirator system 100 is shown according to one embodiment. The respirator system 100 comprises a face mask 101 connected to a blower 108. The face mask 101 includes an air-permeable body 103 made of a filter material 102 designed to filter particulates. Straps 106 help the air-permeable body 103 form a seal with the user's face during use. In some embodiments, a bendable nose strip 104 is provided to enhance a seal with the user's nose during use. The filter material 102 includes a proximal surface 112 that faces the user 150 when in use, and a distal surface 110 that faces away from the user 150 and is exposed to the unfiltered air. The blower 108 is attached by tubing 109 to an opening 107 on the distal surface 110 of the filter material 102. Blower 108 comprises housing 116 containing lumen 118 extending between inlet 120 and outlet 122. In some embodiments, face mask 101 further comprises pressure sensor 130 positioned downstream from outlet 122. Pressure sensor 130 can be placed at any location downstream from outlet 122, such as within tubing 109, adjacent to opening 107, and on the proximal surface 112 of filter material 102. A replaceable filter 114 is engaged to outlet 122 and to the distal end of tubing 109. Fan 124 is positioned within lumen 118 and is configured to push unfiltered air in a proximal direction through filter 114 and tubing 109 towards the user 150 for delivering filtered air to the user 150. Blower 108 can be powered by power source 126. In some embodiments, blower 108 can be activated and modulated by controller 128.

In some embodiments, face mask 101 further comprises inner seal 132, outer seal 134, or both attached to the proximal surface 112 of filter material 102. Inner seal 132 can comprise a relatively soft material (such as silicone, rubber, or foam) having a substantially circular or elliptical shape configured to form a 360-degree air-tight protective seal between the air-permeable body 103 (and opening 107) and a user's nose and mouth when face mask 101 is worn. Inner seal 132 can also comprise wider or thicker regions configured to conform to the shape of a user's face on either side of the user's nose to improve the sealing characteristics of inner seal 132. Outer seal 134 can comprise a relatively soft material (such as silicone, rubber, or foam) attached to the perimeter of face mask 101 and is configured to conform to the contours of a user's face to form an air-tight seal between the air-permeable body 103 and the user's face when face mask 101 is worn. In one embodiment, a mild adhesive may be used in conjunction with inner seal 132, outer seal 134, or both, in order to further improve the quality of the seal between face mask 101 and a user's face. In another embodiment, inner seal 132, outer seal 134, or both may comprise an elastomeric material formulated to produce a sticky or tacky effect, in order to further improve the quality of the seal between the mask and the user's face.

The blower 108 can be configured to operate at a speed which generates a sub-peak inspiratory flow. In one embodiment, the blower speed is preset to at least one sub-peak inspiratory flow based on predicted peak inspiratory flows. The blower 108 can include a switch or dial configured to select a preset blower speed. In one embodiment, a user's inhalation, exhalation, and breathing pauses in-between are monitored by a pressure sensor. For example, during a breathing pause, the blower is the only source affecting the air pressure within the mask, such that the pressure sensor may measure a first positive air pressure within the mask. During the user's inhalation, the blower supplies a positive pressure while the user draws in air to provide a negative pressure, such that the pressure sensor may measure a net negative air pressure in the mask. During the user's exhalation, the blower supplies a positive pressure while the user exhales out air to provide a positive pressure, such that the pressure sensor may measure a net positive air pressure that is greater than the first positive air pressure.

In some embodiments, the blower speed can be adjusted by a printed circuit board (PCB) controller based on the pressure sensor measurements to maintain a sub-peak inspiratory flow. For example, the PCB controller may measure a substantially sinusoidal air pressure pattern within the mask, wherein the substantially sinusoidal pattern has troughs corresponding to the lowest air pressure during peak inspiratory flow, crests corresponding to the highest air pressure during peak expiratory flow, and a substantially steady state between the troughs and crests corresponding to the positive air pressure supplied by the blower. In one embodiment, the pressure sensor measures the average air pressure in the mask in one or more breaths, and the blower speed is set by the PCB controller to a constant speed that generates the average air pressure as the substantially steady state air pressure. In some embodiments, the blower speed is set by the PCB controller to adjust with a user's inspiratory flow as measured by the pressure sensor, such that the blower speed is increased during less pressure and decreased during greater pressure while maintaining a pressure that corresponds to sub-peak inspiratory flow. In some embodiments, the PCB controller measures the time between each trough or the time between each crest as a respiratory cycle time. The blower speed can then be set by the PCB controller to generate an air pressure that is a percentage of the measured air pressure that is greater than the air pressure during peak inspiratory flow within a single respiratory cycle.

Generally, the range of peak inspiratory flows for the 95th percentile minute volume for occupational tasks is estimated to range between 182 and 295 L min⁻¹ (see Caretti D M et. al., Workplace breathing rates: defining anticipated values and ranges for respirator certification testing, 2004 Report ECBC-TR-316, Edgewood Chemical Biological Center, US Army Research. Aberdeen Proving Ground, MD: Development and Engineering Command). In some embodiments, the resting air inflow rate of a user is about 30 L min⁻¹ while the air inflow rate of a user during strenuous activity is about 60 L min⁻¹. In one embodiment, blower speeds are set to generate sub-peak inspiratory flows between about 1 L min⁻¹ and 150 L min⁻¹. In one embodiment, the blower speed is set to generate an airflow of between about 30 and 85 L min⁻¹. In one embodiment, the blower speed is set to generate an airflow of between about 30 and 60 L min⁻¹. In one embodiment, the blower speed is set to generate an airflow of 30 L min⁻¹ or less. In one embodiment, the blower speed is set to generate an airflow of 50 L min⁻¹ or less. In one embodiment, the blower speed is set to generate an airflow of 70 L min⁻¹ or less. In one embodiment, the blower speed is set to generate an airflow of 85 L min⁻¹ or less. In one embodiment, the blower speed is set to generate an airflow of 150 L min⁻¹ or less. The blower speed can be preset to provide a minimum air flow of about 1 L min⁻¹.

In one embodiment, the blower speed is set to adjust the air pressure in the mask by a percentage of the absolute value of the air pressure measured at a user's peak inspiratory flow, average inspiratory flow, or instant inspiratory flow. For example, the blower speed can be set to adjust the air pressure to about 10%, 20%, 30%, 40%, 50%, 60%, 70%, 80%, or 90% of the absolute value of the air pressure at a user's peak inspiratory flow, average inspiratory flow, or instant inspiratory flow. In one embodiment, the blower speed is set to adjust the air pressure in the mask to a preset amount more negative than the air pressure measured at a user's peak inspiratory flow, average inspiratory flow, or instant inspiratory flow while maintaining a net negative air pressure. For example, the blower speed can be set to adjust the air pressure in the mask during a user's peak inspiratory flow, average inspiratory flow, or instant inspiratory flow by about minus 0.1 cmH₂O, 1 cmH₂O, 2 cmH₂O, 3 cmH₂O, 4 cmH₂O, 5 cmH₂O, or less, while maintaining a net negative air pressure. The blower speed can be preset to generate a minimum air pressure of about minus 0.1 cmH₂O.

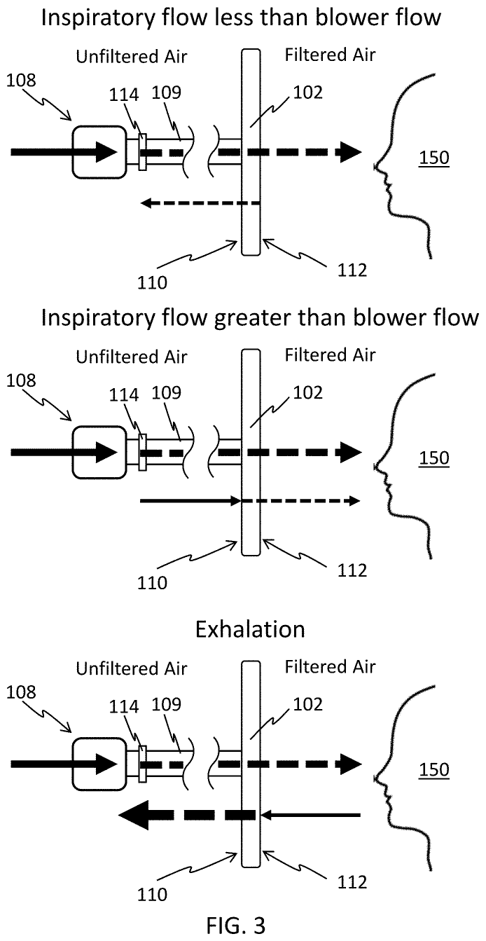

Advantageously, the blower delivers an uninterrupted flow of filtered air that is restricted to be lower than the peak inspiratory flow of a user (FIG. 3, top). During inhaled flow that is higher than the blower flow, the user will draw in the additional air through the filter material of the mask, ensuring all inhaled air is filtered (FIG. 3, middle). The battery life of the blower is thereby extended because the blower is prevented from having to provide a flow speed greater than peak inspiratory flow speed and extends the life of the battery for the blower over a longer period of time. The uninterrupted flow of filtered air also maintains a positive pressure within the mask in-between the user's inhalation and exhalation, preventing unfiltered air from entering. Further, during exhalation, when the flow from the blower is flowing out through the mask (along with the exhaled air), the higher flow of filtered air pushes the particles off the mask, extending its life (FIG. 3, bottom).

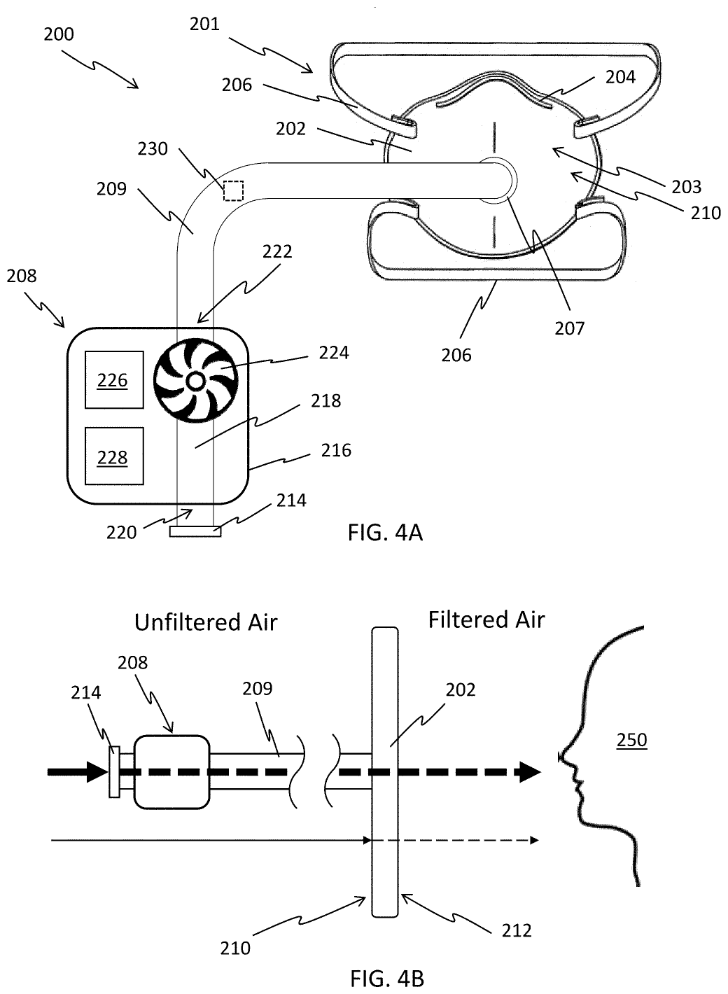

With reference now to FIGS. 4A and 4B, a respirator system 200 is shown according to one embodiment. The respirator system 200 comprises a face mask 201 connected to a blower 208. The face mask 201 includes an air-permeable body 203 made of a filter material 202 designed to filter particulates. Straps 206 help the air-permeable body 203 form a seal with the user's face during use. In some embodiments, a bendable nose strip 204 is provided to enhance a seal with the user's nose during use. The filter material 202 includes a proximal surface 212 that faces the user 250 when in use, and a distal surface 210 that faces away from the user 250 and is exposed to the unfiltered air. A blower 208 is attached by tubing 209 to an opening 207 on the distal surface 210 of the filter material 202. Blower 208 comprises housing 216 containing lumen 218 extending between inlet 220 and outlet 222. A replaceable filter 214 is engaged to inlet 220 and can be exchanged without disconnecting blower 208 from tubing 209. Fan 224 is positioned within lumen 218 and is configured to pull unfiltered air through filter 214 and push filtered air in a proximal direction through tubing 209 towards the user 250 for delivering filtered air to the user 250. Blower 208 can be powered by power source 226. In some embodiments, blower 208 can be activated and modulated by controller 228.

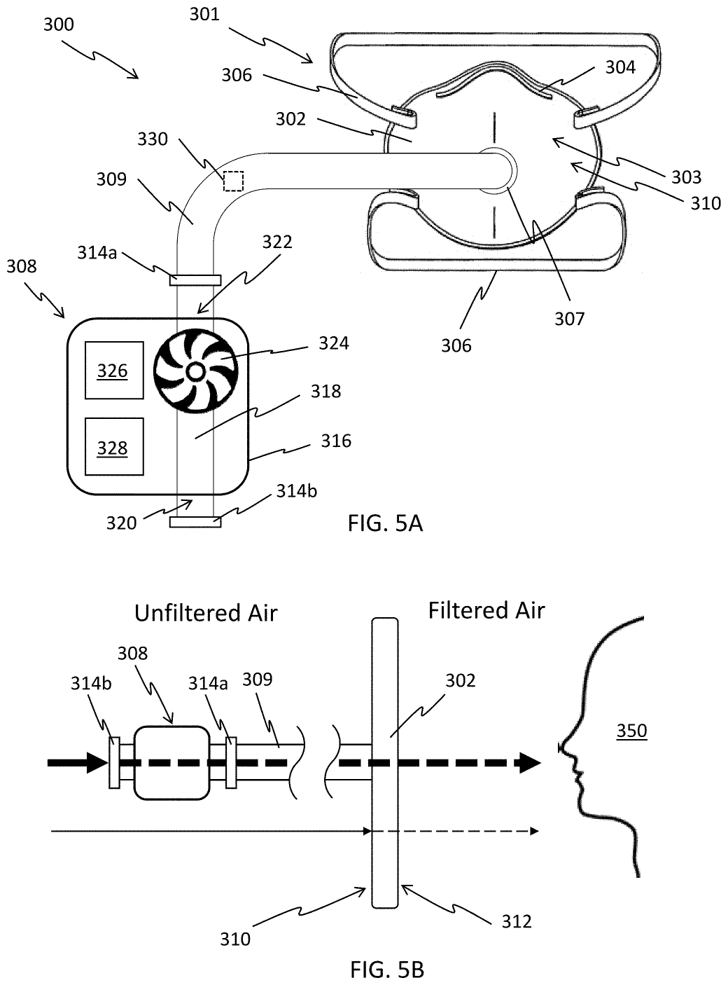

With reference now to FIGS. 5A and 5B, a respirator system 300 is shown according to one embodiment. The respirator system 300 comprises a face mask 301 connected to a blower 308. The face mask 301 includes an air-permeable body 303 made of a filter material 302 designed to filter particulates. Straps 306 help the air-permeable body 303 form a seal with the user's face during use. In some embodiments, a bendable nose strip 304 is provided to enhance a seal with the user's nose during use. The filter material 302 includes a proximal surface 312 that faces the user 350 when in use, and a distal surface 310 that faces away from the user 350 and is exposed to the unfiltered air. A blower 308 is attached by tubing 309 to an opening 307 on the distal surface 310 of the filter material 302. Blower 308 comprises housing 316 containing lumen 318 extending between inlet 320 and outlet 322. A replaceable filter 314a is engaged to outlet 322 and to the distal end of tubing 309, and a replaceable filter 314b is engaged to inlet 320. A dual filter design facilitates exchanging filter 314b while maintaining filtration in filter 314a. Fan 324 is positioned within lumen 318 and is configured to pull unfiltered air through filter 314b and push filtered air in a proximal direction through filter 314a and tubing 309 towards the user 350 for delivering filtered air to the user 350. Blower 308 can be powered by power source 326. In some embodiments, blower 308 can be activated and modulated by controller 328.

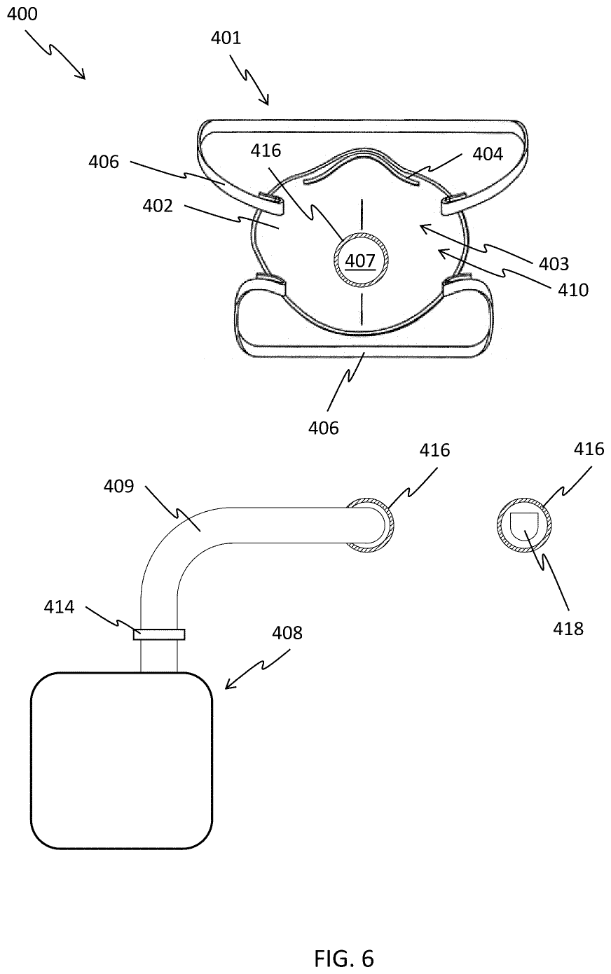

With reference now to FIG. 6, a modular respirator system 400 is shown according to one embodiment. The modular respirator system 400 comprises a face mask 401 connected to a blower 408. The face mask 401 includes an air-permeable body 403 made of a filter material 402 designed to filter particulates. Straps 406 help the air-permeable body 403 form a seal with the user's face during use. In some embodiments, a bendable nose strip 404 is provided to enhance a seal with the user's nose during use. The filter material 402 includes a proximal surface 412 that faces a user when in use, and a distal surface 410 that faces away from a user and is exposed to the unfiltered air. Distal surface 410 has an opening 407 that extends through proximal surface 412. Opening 407 includes engagement 416 configured to releasably attach to compatible engagements 416. Exemplary engagements 416 include but are not limited to: threaded engagements, twist lock engagements, friction fit engagements, magnetic engagements, slotted engagements, clamp engagements, and the like. Engagement 416 can be supplemented with an O-ring, rubber flap, or other mechanism configured to increase air-tightness and prevent the entry of unfiltered air.

Face mask 401 can accept any suitable module attachable to engagement 416. For example, a blower module 408 having at least one filter 414 and tubing 409 with engagement 416 positioned at the proximal end of tubing 409 can be releasably attached to opening 407 of face mask 401 to provide a sub-peak inspiratory flow, as described elsewhere herein. In another example, a one-way valve module 418 having an engagement 416 can be releasably attached to opening 407 to provide a passive exhalation valve to face mask 401.

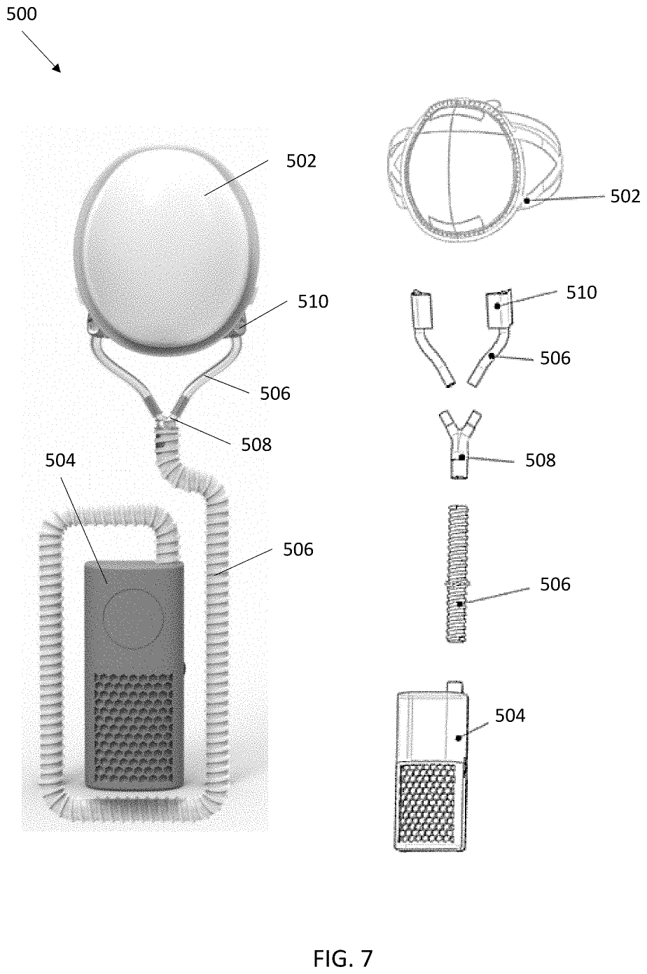

Referring now to FIG. 7, a respirator system 500 is shown according to one embodiment. Respirator system 500 comprises a face mask 502 connected to a fan unit 504 by a plurality of air tubes 506, a Y connector 508, and at least one tube adaptor 510. Fan unit 504 is a portable, lightweight unit (about 300 g) having a convenient size (such as about 150 mm by about 60 mm by about 45 mm). The face mask 502 includes an air-permeable body made of a filter material designed to filter particulates. In various embodiments, the face mask 502 includes one or more of the various features to enhance fit and seal with a user as described elsewhere herein, including but not limited to an inner seal, straps, a bendable nose strip, and the like. The filter material of face mask 502 includes a proximal surface that faces a user when in use and a distal surface that faces away from a user and is exposed to unfiltered air. Unfiltered air is drawn into fan unit 504 where it is filtered and the filtered air is blown through air tube 506 toward face mask 502. Air tube 506 can have any desired dimensions, such as a diameter of about 13 mm and a length between about 500 mm to about 800 mm. In some embodiments, filtered air is blown directly from fan unit 504 through air tube 506 and enters face mask 502 through a single tube adapter 510. In some embodiments, a Y-connector 508 is connected to air tube 506 at a distal end and to additional air tubes 506 at two proximal ends to direct two streams of filtered air into face mask 502 through two tube adapters 510, such that the air is evenly distributed to both sides of a user's face. In some embodiments, face mask 502 comprises entry ports that engage each of the tube adapters 510. In some embodiments, face mask 502 can be any face mask, wherein tube adapters 510 attach to the distal surface facing away from a user by a securing mechanism, such as a clip or screw fitting.

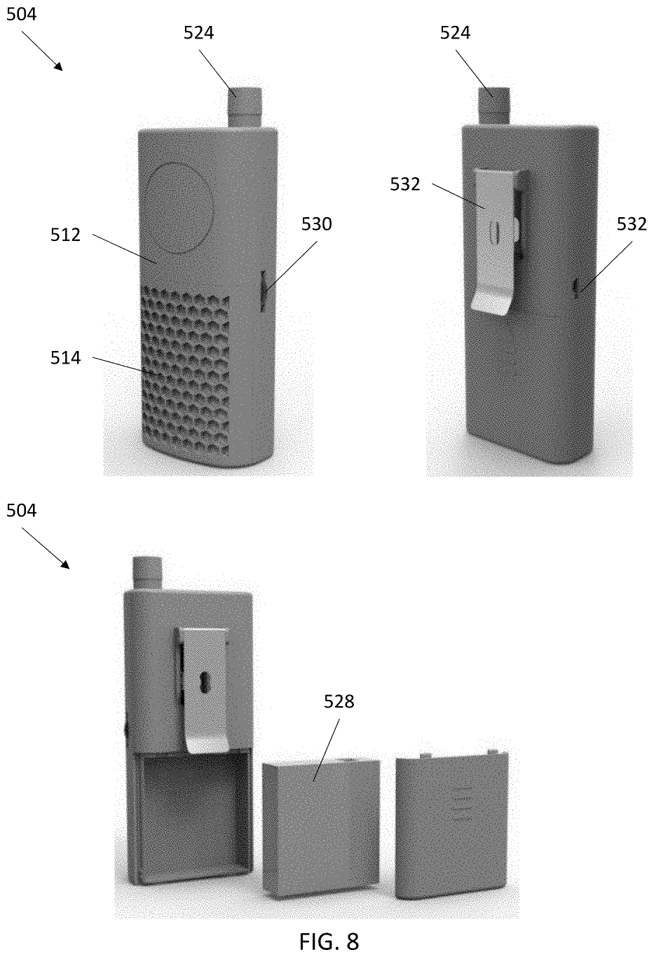

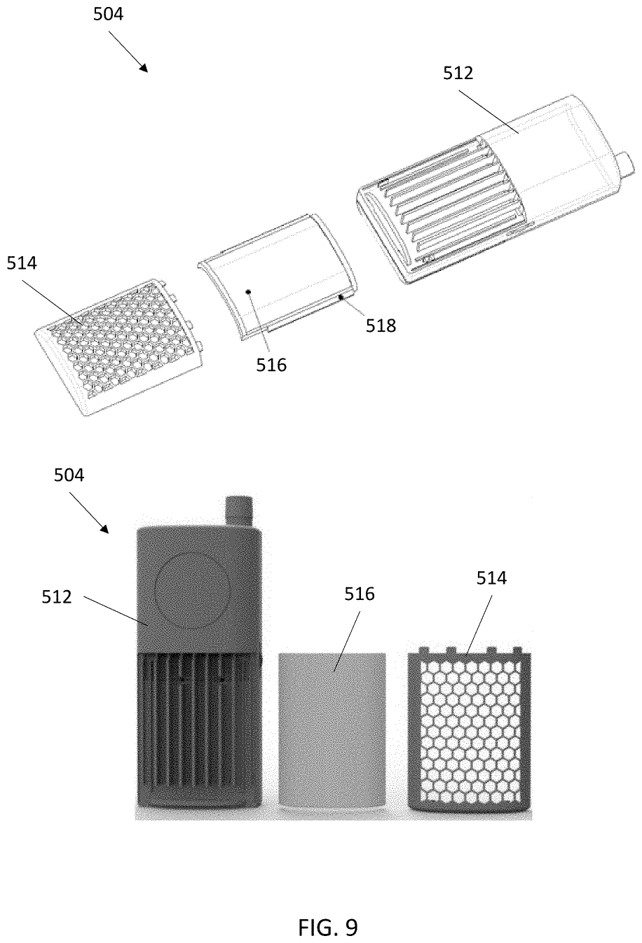

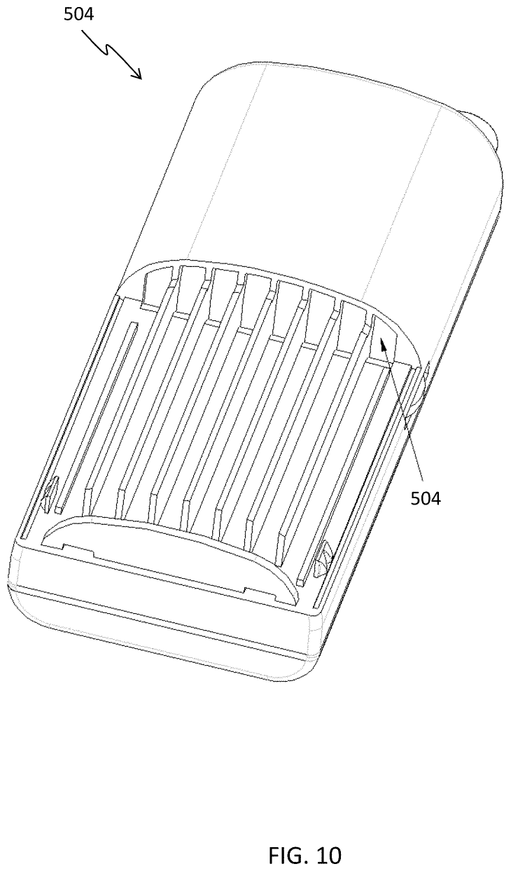

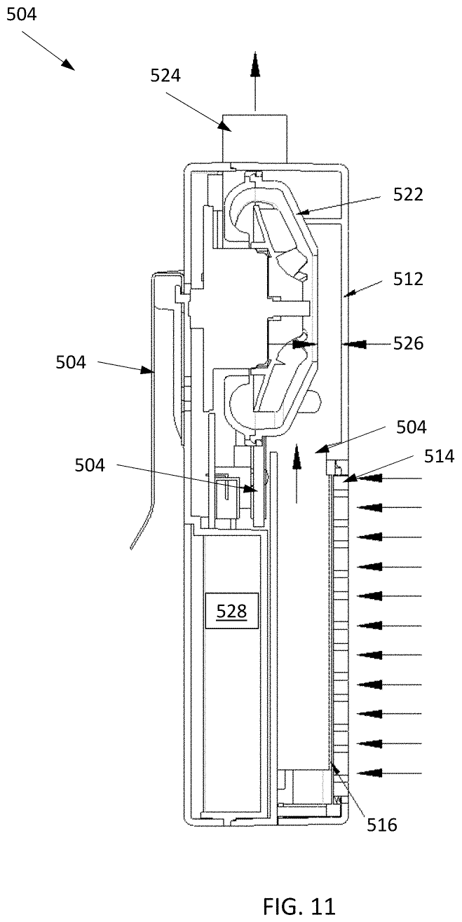

Referring now to FIG. 8, the exterior of fan unit 504 is shown comprising a casing 512, a cover 514, an air outlet 524, a power source 528, a regulating switch 530, and a clip 534. Power source 528 can be a rechargeable power source, such as by way of a charging port 532. Power source 528 can have a high capacity for extended use, such as in the range of 4000 mAh or greater. In some embodiments, fan unit 504 can be activated and fan speed modulated by regulating switch 530. Visible in FIG. 9, removing cover 514 reveals a replaceable filter 516. Filter 516 can have any desired filter efficiency rating, such as a rating of about 95%, about 99%, about 99.97%, or greater. In some embodiments, filter 516 comprises an elastomer gasket 518 on an outer edge to prevent air leaks such that unfiltered air reliably passes through filter 516. In some embodiments, filter 516 rests in a filter tray or cartridge, wherein the filter tray or cartridge comprises an elastomer gasket 518 on an outer edge. As shown in FIG. 10 and FIG. 11, unfiltered air is drawn through cover 514 and through filter 516 by fan 522 to become filtered air. Filtered air is drawn through inlet 504, passes between fan 522 and casing 512 via gap 526. Gap 526 can be any suitable distance, such as a space between about 4 and 6 mm for optimal air flow and noise reduction. It should also be appreciated that air enters fan unit 504 from a side entry port relative to inlet 504. In some embodiments, the side entry port faces a direction that is orthogonal to the direction inlet 504 faces. The side inflow pathway for air permits the use of multiple air ducts to reduce wind resistance and noise, as well as a larger filter surface area. Breathing resistance can be less than 180 Pa, and the combined motor, fan, and wind noise can be less than 50 dB for a user and less than 60 db at a distance of about 1 m. After reaching fan 522, filtered air is blown out through outlet 524 to be channeled to face mask 502 via air tubes 506 as described above to deliver filtered air to a user.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

What is claimed is:

1. A respirator system comprising:
a face mask having an air-permeable body comprising a filter material having a proximal surface, a distal surface, and an opening, the air-permeable body structurally configured for the proximal surface to cover the nose and mouth of a user when worn by the user; and
a blower unit having at least one filter, the blower unit being connected to the opening of the air-permeable body by a length of tubing and configured to generate a sub-peak inspiratory flow of filtered air through the tubing.

2. The respirator system of claim 1, wherein the blower is configured to generate only sub-peak inspiratory flow rates through the filter material.

3. The respirator system of claim 1, wherein a filter is attached proximal to the blower unit and distal to the tubing.

4. The respirator system of claim 1, wherein a filter is attached distal to the blower unit.

5. The respirator system of claim 1, wherein a first filter is attached distal to the blower unit and a second filter is attached proximal to the blower unit and distal to the tubing.

6. The respirator system of claim 1, wherein the tubing is detachable from the opening and replaceable with a one-way valve.

7. The respirator system of claim 1, wherein the blower is configured to generate a sub-peak inspiratory flow of between about 1 L min⁻¹ and 150 L min⁻¹.

8. The respirator system of claim 1, further comprising a pressure sensor.

9. The respirator system of claim 8, wherein the blower has a speed configured as a function of air pressure measured by the pressure sensor during the user's inspiratory flow selected from the group consisting of: peak inspiratory flow, average inspiratory flow, and instant inspiratory flow.

10. The respirator system of claim 9, wherein the blower speed configuration is set to adjust the air pressure in the face mask that is a percentage of the absolute value of the negative air pressure measured during a user's peak inspiratory flow selected from the group consisting of: 10%, 20%, 30%, 40%, 50%, 60%, 70%, 80%, and 90%.

11. The respirator system of claim 9, wherein the blower speed configuration is set to adjust the air pressure in the face mask to a preset amount that is lower than the negative air pressure measured at a user's peak inspiratory flow while maintaining a net negative air pressure, the preset amount being selected from the group consisting of: minus 0.1 cmH₂O, 1 cmH₂O, 2 cmH₂O, 3 cmH₂O, 4 cmH₂O, and 5 cmH₂O.

12. The respirator system of claim 11, wherein the blower speed configuration is set to provide a lower air pressure limit of about minus 0.1 cmH₂O.

13. The respirator system of claim 1, further comprising an inner seal attached to the proximal surface of the air-permeable body around the opening, the inner seal having a substantially circular shape configured to form an air-tight seal around a user's mouth and nose when worn.

14. The respirator system of claim 1, further comprising an outer seal attached to the proximal surface of the air-permeable body, the outer seal forming a perimeter along the air-permeable body configured to form an air-tight seal around a user's face when worn.

15. A respirator system comprising:
a face mask having an air-permeable body comprising a filter material having a proximal surface, a distal surface, and an opening, the air-permeable body structurally configured for the proximal surface to cover the nose and mouth of a user when worn by the user; and
a fan unit having at least one filter, the fan unit being connected to the face mask by a length of tubing and configured to generate an inspiratory flow of filtered air through the tubing between about 30 and 60 L min⁻¹.

16. The respirator system of claim 15, further comprising a Y-connector having a distal end and two proximal ends, wherein the distal end is connected to the length of tubing and the two proximal ends are each connected to an additional length of tubing that is connected to the face mask.

17. The respirator system of claim 15, wherein the fan unit comprises an outer side entry port and an inner air inlet, the outer side entry port having an opening larger than an opening of the inner air inlet.

18. The respirator system of claim 17, wherein the outer side entry port faces a direction orthogonal to a direction the inner air inlet faces.

19. The respirator system of claim 17, wherein the filter has a size that is substantially the same as the opening of the outer side entry port.

20. The respirator system of claim 17, wherein the filter comprises an elastomer gasket on an outer edge.
